(19) United States
(12) Patent Application Publication
(10) Pub. No.: US 2006/0078568 A1
(43) Pub. Date: Apr. 13, 2006

(54) Use of the residues from wine production

(76) Inventors: Gilles Pauly, Nancy (FR); Florence Henry, Villers-Les-Nancy (FR)

Correspondence Address: COGNIS CORPORATION;PATENT DEPARTMENT, 300 BROOKSIDE AVENUE, AMBLER, PA, 19002, US

(21) Appl. No.: 11/229,295

(22) Filed: Sep. 16, 2005

Related U.S. Application Data
(63) Continuation of application No. 10/399,593, filed on Jun. 21, 2002, now abandoned.

(30) Foreign Application Priority Data
Dec. 22, 1999 (FR) 99/16868

(51) Int. Cl. A61K 36/87; A61K 36/064

(52) U.S. Cl. 424/195.16; 424/766

(57) ABSTRACT

A cosmetic or pharmaceutical preparation comprising a winemaking residue is comprised of from about 1 to about 10% by weight of polyphenols and from about 25 to about 50% by weight of proteins, based on the dry residue weight. The press residues obtained in the flocculation of the fermented grape juice contain synergistic mixtures of polyphenols and proteins from the yeasts and increase the efficacy of the cosmetic or pharmaceutical preparations into which they are added for countering the effects of skin aging and damage to fibroblasts and kerotinocytes caused by UV-A and UV-B radiation.

### RELATED APPLICATIONS

This application is a continuation of U.S. application Ser. No. 10/399,593 filed Jun. 21, 2002, which was filed under 35 U.S.C. § 371 claiming priority from PCT/EP00/12650 filed Dec. 13, 2000, claiming priority from French Application Serial No. FR 99/16868 filed Dec. 22, 1999, the entire contents of each application are incorporated herein by reference.

### FIELD OF THE INVENTION

This invention relates generally to cosmetics and more particularly to the use of residues from winemaking for the production of cosmetic and/or pharmaceutical preparations.

### BACKGROUND OF THE INVENTION

Even in ancient times, the juice of the grape was known for its medicinal properties and not just its stimulating effect. However, the realization that the polyphenols present in wine, as natural radical trappers, actually have a positive effect on health is the result of research carried out in this century. This effect is based on the following reaction: 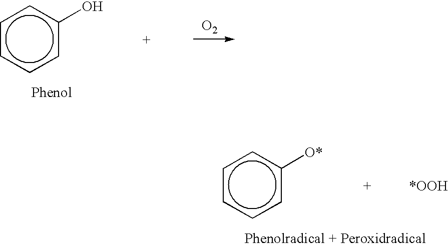
The phenol radical possesses particularly high stability through mesomeric stabilization. Accordingly, cosmetic chemistry has for some time used polyphenols and their esterification products as additives for care and repair products. The extensive prior art literature on the subject is represented, for example, by EP-A1 0692480 A1 (Berkem), EP-A2 0774249 (Unilever), EP-A2 0781544 (Nikka), EP-A1 0842938 (L'Oréal), WO 94/29404 (Ovi) and U.S. Pat. No. 4,698,360 (Horphag). However, it has now been found that the antioxidative and cell-stimulating effect of known polyphenols is subject to major structural variations. Because of this, the substances have to be used in high concentrations which adds significantly to the cost of the formulations.

Accordingly, there is much interest in natural active-substance mixtures which develop a comparable cosmetic effect, but in far smaller quantities. In particular, there is a demand for active substances with anti-inflammatory properties which would activate special repair and detoxification enzymes (for example glutathione-S-transferase), stimulate or regulate cell growth, influence the metabolic activity of fibroblasts or keratinocytes and could thus be used with advantage for the production of cosmetic and/or pharmaceutical preparations, especially skin and hair treatment preparations and sun protection products, without unwanted side effects, even in sensitive users. The problem addressed by the present invention was to provide active substances with the described complex performance profile.

### DESCRIPTION OF THE INVENTION

The present invention relates to the use of residues from winemaking as active-substance compositions for the production of cosmetic and/or pharmaceutical preparations.

It has surprisingly been found that the residues which accumulate in the winemaking process solve the complex problem stated above very effectively. The invention is based on the observation that the press residues obtained in the flocculation of the fermented grape juice contain synergistic mixtures of polyphenols and proteins from the yeasts used, more particularly mannoproteins, which are present quite predominantly as association complexes and develop greater cosmetic or physiological activity than the sum of the individual constituents.

Accordingly, the present invention relates to the manifold use of residues from winemaking as active-substance compositions for the production of cosmetic and/or pharmaceutical preparations
as skin and hair care agents, particularly against stress;
as anti-inflammatory agents;
as antioxidants;
as skin rejuvenating agents, particularly against wrinkles and/or ageing marks;
as agents against fibroblast and/or keratinocyte damage by UV-A and UV-B radiation, more particularly by UV-B radiation;
as agents for stimulating or regulating the formation of skin cells and
as agents for stimulating skin detoxification enzymes, especially glutathione-S-transferase.

### DETAILED DESCRIPTION OF THE INVENTION

Active-Substance Composition

The winemaking process involves a number of steps. After the grape juice has been pressed from the skins and stalks, the must is separated from suspended particles (“preclarified”) and, optionally after the addition of sugar (“chaptalization”), is pumped into vats for fermentation. The yeasts present on the berries or rather the enzymes present in those yeasts convert the grape sugar present in the must into ethanol and carbon dioxide. Fermentation is optionally supported by the addition of yeasts. On completion of the first fermentation, which generally takes 1 to 3 weeks, the “second” (malolactic) fermentation typical above all of red wines may follow although its main function is merely to convert the malic acid present in the must and in the young wine into lactic acid. When the fermentation processes are over, the wine is pumped from the vats which are then left with a residue which contains the active-substance composition to be used in accordance with the invention. The residues to be used in accordance with the invention are rich in polyphenols and proteins from the yeasts used, particularly when beaten eggwhite has been added to the wine for fining, and contain these polyphenols and proteins predominantly in the form of association complexes. One type of the association complexes can be formed when the polyphenols are attached to the cell wall of the yeast by the mannoproteins present.

In the context of the invention, the terms “preparations” and “agents” are synonymous with the term “care preparations”.

Care preparations in the context of the invention are understood to be hair and skin care preparations. These care preparations have inter alia stimulating, regulating, healing and regenerating effects on the skin and hair. Preferred care preparations in the context of the invention are those which have a stimulating and regulating effect on the skin cells and their functions and a regenerating effect on the skin and hair and a protective effect against environmental influences on the skin and hair. Other preferred care preparations in the context of the invention are those which can either ameliorate or cure various diseases of the skin through their various effects on the appearance and function of the skin.

According to the invention, the winemaking residues are used as anti-inflammatory care preparations which are capable of healing or preventing inflammation of the skin. Such inflammation can have various causes. In particular, the preparations according to the invention may be used to treat inflammation induced by UV radiation, contamination of the skin or bacterial and hormonal changes in the skin, for example acne.

According to the invention, the winemaking residues are used as antioxidants which, on the one hand, are capable of disrupting the photochemical reaction chain that is initiated when UV radiation penetrates the skin or which act against any form of skin and hair damage that can be triggered by radical reactions attributable to harmful environmental influences.

According to the invention, the winemaking residues are used against ageing of the skin, above all against all forms of lining and wrinkling and against ageing marks. The uses include the slowing down of skin ageing processes. The ageing signs can have various causes. In particular, they may be caused by UV-induced skin damage. In one particular embodiment, the winemaking residues are used against fibroblast and keratinocyte damage by UV radiation.

Besides the known dihydroxybenzenes (pyrocatechol, resorcinol, hydroquinone), phloroglucinol and pyrogallol, the polyphenols may also be polynuclear complexes, for example the following substances or their oligomerization products: 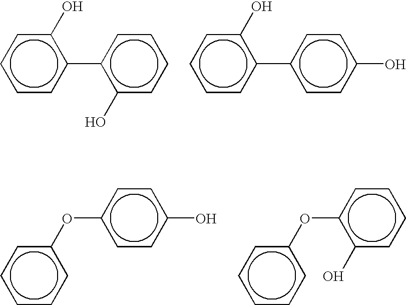
The anthocyanidines, pro-anthocyanidines, flavones, catechols and tannins are particularly preferred. Among the raw materials to be used, residues from the production of red Madeira wine occupy a special position because they have particularly high contents of tannins and oligomeric pro-anthocyanidines.

The proteins present in the mixtures are predominantly enzymes and/or degradation products—i.e. peptide sequences—of enzymes that are present in the cell membrane of the yeasts added to the must during the winemaking process. Accordingly, special constituents are enzymes from Saccharomyces cerevisiae and the degradation products of these enzymes.

Residues containing 1 to 10 and preferably 4 to 6% by weight of polyphenols and 25 to 50 and preferably 30 to 40% by weight of proteins are normally used. The residues are isolated in known manner, for example by means of superdecanters, hydrocyclones or filter presses, optionally in the presence of typical filter aids. The residues normally have a residual moisture content of 5 to 10% by weight.

Commercial Applications

The active-substance compositions to be used in accordance with the invention may be may be used for the production of cosmetic and/or pharmaceutical preparations such as, for example, hair shampoos, hair lotions, foam baths, shower baths, creams, gels, lotions, alcoholic and aqueous/alcoholic solutions, emulsions, wax/fat compounds, stick preparations, powders or ointments. The quantities in which they are used may differ very considerably. In the most simple case, the residues themselves represent the “agent”, in other cases the residues may be added to typical preparations in any quantities. Accordingly, the quantity used may be between 0.1 and 100% by weight and is preferably between 0.5 and 15% by weight and more particularly between 1 and 5% by weight, based on the preparation.

The preparations may contain mild surfactants, oil components, emulsifiers, superfatting agents, pearlizing waxes, consistency factors, thickeners, polymers, silicone compounds, fats, waxes, lecithins, phospholipids, stabilizers, biogenic agents, deodorants, antiperspirants, antidandruff agents, film formers, swelling agents, UV protection factors, antioxidants, hydrotropes, preservatives, insect repellents, self-tanning agents, tyrosine inhibitors (depigmenting agents), solubilizers, perfume oils, dyes and the like as further auxiliaries and additives.

Typical examples of suitable mild, i.e. particularly dermatologically compatible, surfactants are fatty alcohol polyglycol ether sulfates, monoglyceride sulfates, mono- and/or dialkyl sulfosuccinates, fatty acid isethionates, fatty acid sarcosinates, fatty acid taurides, fatty acid glutamates, α-olefin sulfonates, ether carboxylic acids, alkyl oligoglucosides, fatty acid glucamides, alkylamidobetaines and/or protein fatty acid condensates, preferably based on wheat proteins.

Suitable oil components are, for example, Guerbet alcohols based on fatty alcohols containing 6 to 18 and preferably 8 to 10 carbon atoms, esters of linear C₆₋₂₂ fatty acids with linear C₆₋₂₂ fatty alcohols, esters of branched C₆₋₁₃ carboxylic acids with linear C₆₋₂₂ fatty alcohols such as, for example, myristyl myristate, myristyl palmitate, myristyl stearate, myristyl isostearate, myristyl oleate, myristyl behenate, myristyl erucate, cetyl myristate, cetyl palmitate, cetyl stearate, cetyl isostearate, cetyl oleate, cetyl behenate, cetyl erucate, stearyl myristate, stearyl palmitate, stearyl stearate, stearyl isostearate, stearyl oleate, stearyl behenate, stearyl erucate, isostearyl myristate, isostearyl palmitate, isostearyl stearate, isostearyl isostearate, isostearyl oleate, isostearyl behenate, isostearyl oleate, oleyl myristate, oleyl palmitate, oleyl stearate, oleyl isostearate, oleyl oleate, oleyl behenate, oleyl erucate, behenyl myristate, behenyl palmitate, behenyl stearate, behenyl isostearate, behenyl oleate, behenyl behenate, behenyl erucate, erucyl myristate, erucyl palmitate, erucyl stearate, erucyl isostearate, erucyl oleate, erucyl behenate and erucyl erucate. Also suitable are esters of linear C₆₋₂₂ fatty acids with branched alcohols, more particularly 2-ethyl hexanol, esters of hydroxycarboxylic acids with linear or branched C₆₋₂₂ fatty alcohols, more especially Dioctyl Malate, esters of linear and/or branched fatty acids with polyhydric alcohols (for example propylene glycol, dimer diol or trimer triol) and/or Guerbet alcohols, triglycerides based on C₆₋₁₀ fatty acids, liquid mono-, di- and tri-glyceride mixtures based on C₆₋₁₈ fatty acids, esters of C₆₋₂₂ fatty alcohols and/or Guerbet alcohols with aromatic carboxylic acids, more particularly benzoic acid, esters of C₂₋₁₂ dicarboxylic acids with linear or branched alcohols containing 1 to 22 carbon atoms or polyols containing 2 to 10 carbon atoms and 2 to 6 hydroxyl groups, vegetable oils, branched primary alcohols, substituted cyclohexanes, linear and branched C₆₋₂₂ fatty alcohol carbonates, Guerbet carbonates, esters of benzoic acid with linear and/or branched C₆₋₂₂ alcohols (for example Finsolv® TN), linear or branched, symmetrical or nonsymmetrical dialkyl ethers containing 6 to 22 carbon atoms per alkyl group, ring opening products of epoxidized fatty acid esters with polyols, silicone oils and/or aliphatic or naphthenic hydrocarbons, for example squalane, squalene or dialkyl cyclohexanes.

Suitable emulsifiers are, for example, nonionic surfactants from at least one of the following groups:
products of the addition of 2 to 30 moles of ethylene oxide and/or 0 to 5 moles of propylene oxide onto linear C₈₋₂₂ fatty alcohols, C₁₂₋₂₂ fatty acids and alkyl phenols containing 8 to 15 carbon atoms in the alkyl group and alkylamines containing 8 to 22 carbon atoms in the alkyl group;
alkyl and/or alkenyl oligoglycosides containing 8 to 22 carbon atoms in the alkyl group and ethoxylated analogs thereof;
adducts of 1 to 15 moles of ethylene oxide with castor oil and/or hydrogenated castor oil;
adducts of 15 to 60 moles of ethylene oxide with castor oil and/or hydrogenated castor oil;
partial esters of glycerol and/or sorbitan with unsaturated, linear or saturated, branched fatty acids containing 12 to 22 carbon atoms and/or hydroxycarboxylic acids containing 3 to 18 carbon atoms and adducts thereof with 1 to 30 moles of ethylene oxide;
partial esters of polyglycerol (average degree of self-condensation 2 to 8), polyethylene glycol (molecular weight 400 to 5000), trimethylolpropane, pentaerythritol, sugar alcohols (for example sorbitol), alkyl glucosides (for example methyl glucoside, butyl glucoside, lauryl glucoside) and polyglucosides (for example cellulose) with saturated and/or unsaturated, linear or branched fatty acids containing 12 to 22 carbon atoms and/or hydroxycarboxylic acids containing 3 to 18 carbon atoms and adducts thereof with 1 to 30 moles of ethylene oxide;
mixed esters of pentaerythritol, fatty acids, citric acid and fatty alcohol according to DE 11 65 574 PS and/or mixed esters of fatty acids containing 6 to 22 carbon atoms, methyl glucose and polyols, preferably glycerol or polyglycerol,
mono-, di- and trialkyl phosphates and mono-, di- and/or tri-PEG-alkyl phosphates and salts thereof,
wool wax alcohols,
polysiloxane/polyalkyl/polyether copolymers and corresponding derivatives,
polyalkylene glycols and
glycerol carbonate.

The addition products of ethylene oxide and/or propylene oxide with fatty alcohols, fatty acids, alkylphenols or with castor oil are known commercially available products. They are homolog mixtures of which the average degree of alkoxylation corresponds to the ratio between the quantities of ethylene oxide and/or propylene oxide and substrate with which the addition reaction is carried out. C_{12/18} fatty acid monoesters and diesters of adducts of ethylene oxide with glycerol are known as refatting agents for cosmetic formulations from DE 20 24 051 PS.

Alkyl and/or alkenyl oligoglycosides, their production and their use are known from the prior art. They are produced in particular by reacting glucose or oligosaccharides with primary alcohols containing 8 to 18 carbon atoms. So far as the glycoside unit is concerned, both monoglycosides in which a cyclic sugar unit is attached to the fatty alcohol by a glycoside bond and oligomeric glycosides with a degree of oligomerization of preferably up to about 8 are suitable. The degree of oligomerization is a statistical mean value on which the homolog distribution typical of such technical products is based.

Typical examples of suitable partial glycerides are hydroxystearic acid monoglyceride, hydroxystearic acid diglyceride, isostearic acid monoglyceride, isostearic acid diglyceride, oleic acid monoglyceride, oleic acid diglyceride, ricinoleic acid monoglyceride, ricinoleic acid diglyceride, linoleic acid monoglyceride, linoleic acid diglyceride, linolenic acid monoglyceride, linolenic acid diglyceride, erucic acid monoglyceride, erucic acid diglyceride, tartaric acid monoglyceride, tartaric acid diglyceride, citric acid monoglyceride, citric acid diglyceride, malic acid monoglyceride, malic acid diglyceride and technical mixtures thereof which may still contain small quantities of triglyceride from the production process. Addition products of 1 to 30 and preferably 5 to 10 moles of ethylene oxide with the partial glycerides mentioned are also suitable.

Suitable sorbitan esters are sorbitan monoisostearate, sorbitan sesquiisostearate, sorbitan diisostearate, sorbitan triisostearate, sorbitan monooleate, sorbitan sesquioleate, sorbitan dioleate, sorbitan trioleate, sorbitan monoerucate, sorbitan sesquierucate, sorbitan dierucate, sorbitan trierucate, sorbitan monoricinoleate, sorbitan sesquiricinoleate, sorbitan diricinoleate, sorbitan triricinoleate, sorbitan monohydroxystearate, sorbitan sesquihydroxystearate, sorbitan dihydroxystearate, sorbitan trihydroxystearate, sorbitan monotartrate, sorbitan sesquitartrate, sorbitan ditartrate, sorbitan tritartrate, sorbitan monocitrate, sorbitan sesquicitrate, sorbitan dicitrate, sorbitan tricitrate, sorbitan monomaleate, sorbitan sesquimaleate, sorbitan dimaleate, sorbitan trimaleate and technical mixtures thereof. Addition products of 1 to 30 and preferably 5 to 10 moles of ethylene oxide with the sorbitan esters mentioned are also suitable.

Typical examples of suitable polyglycerol esters are Polyglyceryl-2 Dipolyhydroxystearate (Dehymuls® PGPH), Polyglycerin-3-Diisostearate (Lameform® TGI), Polyglyceryl-4 Isostearate (Isolan® GI 34), Polyglyceryl-3 Oleate, Diisostearoyl Polyglyceryl-3 Diisostearate (Isolan® PDI), Polyglyceryl-3 Methylglucose Distearate (Tego Care® 450), Polyglyceryl-3 Beeswax (Cera Bellina®), Polyglyceryl-4 Caprate (Polyglycerol Caprate T2010/90), Polyglyceryl-3 Cetyl Ether (Chimexane® NL), Polyglyceryl-3 Distearate (Cremophor® GS 32) and Polyglyceryl Polyricinoleate (Admul® WOL 1403), Polyglyceryl Dimerate Isostearate and mixtures thereof.

Examples of other suitable polyolesters are the mono-, di- and triesters of trimethylolpropane or pentaerythritol with lauric acid, coconut fatty acid, tallow fatty acid, palmitic acid, stearic acid, oleic acid, behenic acid and the like optionally reacted with 1 to 30 moles of ethylene oxide.

Other suitable emulsifiers are zwitterionic surfactants. Zwitterionic surfactants are surface-active compounds which contain at least one quaternary ammonium group and at least one carboxylate and one sulfonate group in the molecule. Particularly suitable zwitterionic surfactants are the so-called betaines, such as the N-alkyl-N,N-dimethyl ammonium glycinates, for example cocoalkyl dimethyl ammonium glycinate, N-acylaminopropyl-N,N-dimethyl ammonium glycinates, for example cocoacylaminopropyl dimethyl ammonium glycinate, and 2-alkyl-3-carboxymethyl-3-hydroxyethyl imidazolines containing 8 to 18 carbon atoms in the alkyl or acyl group and cocoacylaminoethyl hydroxyethyl carboxymethyl glycinate. The fatty acid amide derivative known under the CTFA name of Cocamidopropyl Betaine is particularly preferred. Ampholytic surfactants are also suitable emulsifiers. Ampholytic surfactants are surface-active compounds which, in addition to a C_{8/18} alkyl or acyl group, contain at least one free amino group and at least one —COOH— or —SO₃H— group in the molecule and which are capable of forming inner salts. Examples of suitable ampholytic surfactants are N-alkyl glycines, N-alkyl propionic acids, N-alkylaminobutyric acids, N-alkyliminodipropionic acids, N-hydroxyethyl-N-alkylamidopropyl glycines, N-alkyl taurines, N-alkyl sarcosines, 2-alkylaminopropionic acids and alkylaminoacetic acids containing around 8 to 18 carbon atoms in the alkyl group. Particularly preferred ampholytic surfactants are N-cocoalkylaminopropionate, cocoacylaminoethyl aminopropionate and C_{12/18} acyl sarcosine.

Finally, cationic surfactants are also suitable emulsifiers, those of the esterquat type, preferably methyl-quaternized difatty acid triethanolamine ester salts, being particularly preferred.

Superfatting agents may be selected from such substances as, for example, lanolin and lecithin and also polyethoxylated or acylated lanolin and lecithin derivatives, polyol fatty acid esters, monoglycerides and fatty acid alkanolamides, the fatty acid alkanolamides also serving as foam stabilizers.

Suitable pearlizing waxes are, for example, alkylene glycol esters, especially ethylene glycol distearate; fatty acid alkanolamides, especially cocofatty acid diethanolamide; partial glycerides, especially stearic acid monoglyceride; esters of polybasic, optionally hydroxysubstituted carboxylic acids with fatty alcohols containing 6 to 22 carbon atoms, especially long-chain esters of tartaric acid; fatty compounds, such as for example fatty alcohols, fatty ketones, fatty aldehydes, fatty ethers and fatty carbonates which contain in all at least 24 carbon atoms, especially laurone and distearylether; fatty acids, such as stearic acid, hydroxystearic acid or behenic acid, ring opening products of olefin epoxides containing 12 to 22 carbon atoms with fatty alcohols containing 12 to 22 carbon atoms and/or polyols containing 2 to 15 carbon atoms and 2 to 10 hydroxyl groups and mixtures thereof.

The consistency factors mainly used are fatty alcohols or hydroxyfatty alcohols containing 12 to 22 and preferably 16 to 18 carbon atoms and also partial glycerides, fatty acids or hydroxyfatty acids. A combination of these substances with alkyl oligoglucosides and/or fatty acid N-methyl glucamides of the same chain length and/or polyglycerol poly-12-hydroxystearates is preferably used.

Suitable thickeners are, for example, Aerosil® types (hydrophilic silicas), polysaccharides, more especially xanthan gum, guar-guar, agar-agar, alginates and tyloses, carboxymethyl cellulose and hydroxyethyl cellulose, also relatively high molecular weight polyethylene glycol mono-esters and diesters of fatty acids, polyacrylates (for example Carbopols® [Goodrich] or Synthalens® [Sigma]), polyacrylamides, polyvinyl alcohol and polyvinyl pyrrolidone, surfactants such as, for example, ethoxylated fatty acid glycerides, esters of fatty acids with polyols, for example pentaerythritol or trimethylol propane, narrow-range fatty alcohol ethoxylates or alkyl oligoglucosides and electrolytes, such as sodium chloride and ammonium chloride.

Suitable cationic polymers are, for example, cationic cellulose derivatives such as, for example, the quaternized hydroxyethyl cellulose obtainable from Amerchol under the name of Polymer JR 400®), cationic starch, copolymers of diallyl ammonium salts and acrylamides, quaternized vinyl pyrrolidone/vinyl imidazole polymers such as, for example, Luviquat® (BASF), condensation products of polyglycols and amines, quaternized collagen polypeptides such as, for example, Lauryidimonium Hydroxypropyl Hydrolyzed Collagen (Lamequat® L, Grünau), quaternized wheat polypeptides, polyethyleneimine, cationic silicone polymers such as, for example, Amodimethicone, copolymers of adipic acid and dimethylamino-hydroxypropyl diethylenetriamine (Cartaretine®, Sandoz), copolymers of acrylic acid with dimethyl diallyl ammonium chloride (Merquat® 550, Chemviron), polyaminopolyamides as described, for example, in FR 2 252 840 A and crosslinked water-soluble polymers thereof, cationic chitin derivatives such as, for example, quaternized chitosan, optionally in micro-crystalline distribution, condensation products of dihaloalkylene, for example dibromobutane, with bis-dialkylamines, for example bis-dimethylamino-1,3-propane, cationic guar gum such as, for example, Jaguar®CBS, Jaguar®C-17, Jaguar®C-16 of Celanese, quaternized ammonium salt polymers such as, for example, Mirapol® A-15, Mirapol® AD-1, Mirapol® AZ-1 of Miranol.

Suitable anionic, zwitterionic, amphoteric and nonionic polymers are, for example, vinyl acetate/crotonic acid copolymers, vinyl pyrrolidone/vinyl acrylate copolymers, vinyl acetate/butyl maleate/isobornyl acrylate copolymers, methyl vinylether/maleic anhydride copolymers and esters thereof, uncrosslinked and polyol-crosslinked polyacrylic acids, acrylamidopropyl trimethylammonium chloride/acrylate copolymers, octylacrylamide/methyl methacrylate/tert.-butylaminoethyl methacrylate/2-hydroxypropyl methacrylate copolymers, polyvinyl pyrrolidone, vinyl pyrrolidone/vinyl acetate copolymers, vinyl pyrrolidone/dimethylaminoethyl methacrylate/vinyl caprolactam terpolymers and optionally derivatized cellulose ethers and silicones.

Suitable silicone compounds are, for example, dimethyl polysiloxanes, methylphenyl polysiloxanes, cyclic silicones and amino-, fatty acid-, alcohol-, polyether-, epoxy-, fluorine-, glycoside- and/or alkyl-modified silicone compounds which may be both liquid and resin-like at room temperature. Other suitable silicone compounds are simethicones which are mixtures of dimethicones with an average chain length of 200 to 300 dimethylsiloxane units and hydrogenated silicates. A detailed overview of suitable volatile silicones can be found in Todd et al. in Cosm. Toil. 91, 27 (1976).

Typical examples of fats are glycerides while suitable waxes are inter alia natural waxes such as, for example, candelilla wax, carnauba wax, Japan wax, espartograss wax, cork wax, guaruma wax, rice oil wax, sugar cane wax, ouricury wax, montan wax, beeswax, shellac wax, spermaceti, lanolin (wool wax), uropygial fat, ceresine, ozocerite (earth wax), petrolatum, paraffin waxes, microwaxes; chemically modified waxes (hard waxes) such as, for example, montan ester waxes, sasol waxes, hydrogenated jojoba waxes and synthetic waxes such as, for example, polyalkylene waxes and polyethylene glycol waxes. Besides the fats, other suitable additives are fat-like substances, such as lecithins and phospholipids. Lecithins are known among experts as glycerophospholipids which are formed from fatty acids, glycerol, phosphoric acid and choline by esterification. Accordingly, lecithins are also frequently referred to by experts as phosphatidyl cholines (PCs). Examples of natural lecithins are the kephalins which are also known as phosphatidic acids and which are derivatives of 1,2-diacyl-sn-glycerol-3-phosphoric acids. By contrast, phospholipids are generally understood to be mono- and preferably diesters of phosphoric acid with glycerol (glycerophosphates) which are normally classed as fats. Sphingosines and sphingolipids are also suitable.

Metal salts of fatty acids such as, for example, magnesium, aluminium and/or zinc stearate or ricinoleate may be used as stabilizers.

In the context of the invention, biogenic agents are, for example, tocopherol, tocopherol acetate, tocopherol palmitate, ascorbic acid, deoxyribonucleic acid, retinol, bisabolol, allantoin, phytantriol, panthenol, AHA acids, amino acids, ceramides, pseudoceramides, essential oils, other plant extracts and vitamin complexes.

Cosmetic deodorants counteract, mask or eliminate body odors. Body odors are formed through the action of skin bacteria on apocrine perspiration which results in the formation of unpleasant-smelling degradation products. Accordingly, deodorants contain active principles which act as germ inhibitors, enzyme inhibitors, odor absorbers or odor maskers.

Basically, suitable germ inhibitors are any substances which act against gram-positive bacteria such as, for example, 4-hydroxybenzoic acidand salts and esters thereof, N-(4-chlorophenyl)-N′-(3,4-dichlorophenyl)-urea, 2,4,4′-trichloro-2′-hydroxydiphenylether (triclosan), 4-chloro-3,5-dimethylphenol, 2,2′-methylene-bis-(6-bromo-4-chlorophenol), 3-methyl-4-(1-methylethyl)-phenol, 2-benzyl-4-chlorophenol, 3-(4-chlorophenoxy)-propane-1,2-diol, 3-iodo-2-propinyl butyl carbamate, chlorhexidine, 3,4,4′-trichlorocarbanilide (TTC), antibacterial perfumes, thymol, thyme oil, eugenol, clove oil, menthol, mint oil, farnesol, phenoxyethanol, glycerol monolaurate (GML), diglycerol monocaprate (DMC), salicylic acid-N-alkylamides such as, for example, salicylic acid-n-octyl amide or salicylic acid-n-decyl amide.

Suitable enzyme inhibitors are, for example, esterase inhibitors. Esterase inhibitors are preferably trialkyl citrates, such as trimethyl citrate, tripropyl citrate, triisopropyl citrate, tributyl citrate and, in particular, triethyl citrate (Hydagen® CAT, Henkel KGaA, Düsseldorf, FRG). Esterase inhibitors inhibit enzyme activity and thus reduce odor formation. Other esterase inhibitors are sterol sulfates or phosphates such as, for example, lanosterol, cholesterol, campesterol, stigmasterol and sitosterol sulfate or phosphate, dicarboxylic acids and esters thereof, for example glutaric acid, glutaric acid monoethyl ester, glutaric acid diethyl ester, adipic acid, adipic acid monoethyl ester, adipic acid diethyl ester, malonic acid and malonic acid diethyl ester, hydroxycarboxylic acids and esters thereof, for example citric acid, malic acid, tartaric acid or tartaric acid diethyl ester, and zinc glycinate.

Suitable odor absorbers are substances which are capable of absorbing and largely retaining the odor-forming compounds. They reduce the partial pressure of the individual components and thus also reduce the rate at which they spread. An important requirement in this regard is that perfumes must remain unimpaired. Odor absorbers are not active against bacteria. They contain, for example, a complex zinc salt of ricinoleic acid or special perfumes of largely neutral odor known to the expert as “fixateurs” such as, for example, extracts of ladanum or styrax or certain abietic acid derivatives as their principal component. Odor maskers are perfumes or perfume oils which, besides their odor-masking function, impart their particular perfume note to the deodorants. Suitable perfume oils are, for example, mixtures of natural and synthetic perfumes. Natural perfumes include the extracts of blossoms, stems and leaves, fruits, fruit peel, roots, woods, herbs and grasses, needles and branches, resins and balsams. Animal raw materials, for example civet and beaver, may also be used. Typical synthetic perfume compounds are products of the ester, ether, aldehyde, ketone, alcohol and hydrocarbon type. Examples of perfume compounds of the ester type are benzyl acetate, p-tert.butyl cyclohexylacetate, linalyl acetate, phenyl ethyl acetate, linalyl benzoate, benzyl formate, allyl cyclohexyl propionate, styrallyl propionate and benzyl salicylate. Ethers include, for example, benzyl ethyl ether while aldehydes include, for example, the linear alkanals containing 8 to 18 carbon atoms, citral, citronellal, citronellyloxyacetaldehyde, cyclamen aldehyde, hydroxycitronellal, lilial and bourgeonal. Examples of suitable ketones are the ionones and methyl cedryl ketone. Suitable alcohols are anethol, citronellol, eugenol, isoeugenol, geraniol, linalool, phenylethyl alcohol and terpineol. The hydrocarbons mainly include the terpenes and balsams. However, it is preferred to use mixtures of different perfume compounds which, together, produce an agreeable fragrance. Other suitable perfume oils are essential oils of relatively low volatility which are mostly used as aroma components. Examples are sage oil, camomile oil, clove oil, melissa oil, mint oil, cinnamon leaf oil, lime-blossom oil, juniper berry oil, vetiver oil, olibanum oil, galbanum oil, ladanum oil and lavendin oil. The following are preferably used either individually or in the form of mixtures: bergamot oil, dihydromyrcenol, lilial, lyral, citronellol, phenylethyl alcohol, α-hexylcinnamaldehyde, geraniol, benzyl acetone, cyclamen aldehyde, linalool, Boisambrene Forte, Ambroxan, indole, hedione, sandelice, citrus oil, mandarin oil, orange oil, allylamyl glycolate, cyclovertal, lavendin oil, clary oil, β-damascone, geranium oil bourbon, cyclohexyl salicylate, Vertofix Coeur, Iso-E-Super, Fixolide NP, evernyl, iraldein gamma, phenylacetic acid, geranyl acetate, benzyl acetate, rose oxide, romilat, irotyl and floramat.

Antiperspirants reduce perspiration and thus counteract underarm wetness and body odor by influencing the activity of the eccrine sweat glands. Aqueous or water-free antiperspirant formulations typically contain the following ingredients:
astringent active principles,
oil components,
nonionic emulsifiers,
co-emulsifiers,
consistency factors,
auxiliaries in the form of, for example, thickeners or complexing agents and/or
nonaqueous solvents such as, for example, ethanol, propylene glycol and/or glycerol.

Suitable astringent active principles of antiperspirants are, above all, salts of aluminium, zirconium or zinc. Suitable antihydrotic agents of this type are, for example, aluminium chloride, aluminium chlorohydrate, aluminium dichlorohydrate, aluminium sesquichlorohydrate and complex compounds thereof, for example with 1,2-propylene glycol, aluminium hydroxyallantoinate, aluminium chloride tartrate, aluminium zirconium trichlorohydrate, aluminium zirconium tetrachlorohydrate, aluminium zirconium pentachlorohydrate and complex compounds thereof, for example with amino acids, such as glycine.

Oil-soluble and water-soluble auxiliaries typically encountered in antiperspirants may also be present in relatively small amounts. Oil-soluble auxiliaries such as these include, for example,
inflammation-inhibiting, skin-protecting or pleasant-smelling essential oils,
synthetic skin-protecting agents and/or
oil-soluble perfume oils.

Typical water-soluble additives are, for example, preservatives, water-soluble perfumes, pH adjusters, for example buffer mixtures, water-soluble thickeners, for example water-soluble natural or synthetic polymers such as, for example, xanthan gum, hydroxyethyl cellulose, polyvinyl pyrrolidone or high molecular weight polyethylene oxides.

Suitable antidandruff agents are Octopirox® (1-hydroxy-4-methyl-6-(2,4,4-trimethylpentyl)-2-(1H)-pyridinone monoethanolamine salt), Baypival, Piroctone Olamine, Ketoconazole® (4-acetyl-1-{4-[2-(2,4-dichlorophenyl)r-2-(1 H-imidazol-1-ylmethyl)-1,3-dioxylan-c-4-ylmethoxy-phenyl}-piperazine, selenium disulfide, colloidal sulfur, sulfur polyethylene glycol sorbitan monooleate, sulfur ricinol polyethoxylate, sulfur tar distillate, salicylic acid (or in combination with hexachlorophene), undecylenic acid, monoethanolamide sulfosuccinate Na salt, Lamepon® UD (protein/undecylenic acid condensate), zinc pyrithione, aluminium pyrithione and magnesium pyrithione/dipyrithione magnesium sulfate.

Standard film formers are, for example, chitosan, microcrystalline chitosan, quaternized chitosan, polyvinyl pyrrolidone, vinyl pyrrolidone/vinyl acetate copolymers, polymers of the acrylic acid series, quaternary cellulose derivatives, collagen, hyaluronic acid and salts thereof and similar compounds.

Suitable swelling agents for aqueous phases are montmorillonites, clay minerals, Pemulen and alkyl-modified Carbopol types (Goodrich). Other suitable polymers and swelling agents can be found in R. Lochhead's review in Cosm. Toil. 108, 95 (1993).

UV protection factors in the context of the invention are, for example, organic substances (light filters) which are liquid or crystalline at room temperature and which are capable of absorbing ultraviolet or infrared radiation and of releasing the energy absorbed in the form of longer-wave radiation, for example heat. UV-B filters can be oil-soluble or water-soluble. The following are examples of oil-soluble substances:
3-benzylidene camphor or 3-benzylidene norcamphor and derivatives thereof, for example 3-(4-methylbenzylidene)-camphor as described in EP-B1 0693471;
4-aminobenzoic acid derivatives, preferably 4-(dimethylamino)-benzoic acid-2-ethylhexyl ester, 4-(dimethylamino)-benzoic acid-2-octyl ester and 4-(dimethylamino)-benzoic acid amyl ester;
esters of cinnamic acid, preferably 4-methoxycinnamic acid-2-ethylhexyl ester, 4-methoxycinnamic acid propyl ester, 4-methoxycinnamic acid isoamyl ester, 2-cyano-3,3-phenylcinnamic acid-2-ethylhexyl ester (Octocrylene);
esters of salicylic acid, preferably salicylic acid-2-ethylhexyl ester, salicylic acid-4-isopropylbenzyl ester, salicylic acid homomenthyl ester;
derivatives of benzophenone, preferably 2-hydroxy-4-methoxybenzo-phenone, 2-hydroxy-4-methoxy-4′-methylbenzophenone, 2,2′-dihydroxy-4-methoxybenzophenone;
esters of benzalmalonic acid, preferably 4-methoxybenzmalonic acid di-2-ethylhexyl ester;
triazine derivatives such as, for example, 2,4,6-trianilino-(p-carbo-2′-ethyl-1′-hexyloxy)-1,3,5-triazine and Octyl Triazone as described in EP 0818450 A1 or Dioctyl Butamido Triazone (Uvasorb® HEB);
propane-1,3-diones such as, for example, 1-(4-tert.butylphenyl)-3-(4′-methoxyphenyl)-propane-1,3-dione;
ketotricyclo(5.2.1.0)decane derivatives as described in EP 0694521 B1.

Suitable water-soluble substances are
2-phenylbenzimidazole-5-sulfonic acid and alkali metal, alkaline earth metal, ammonium, alkylammonium, alkanolammonium and glucammonium salts thereof;
sulfonic acid derivatives of benzophenones, preferably 2-hydroxy-4-methoxybenzophenone-5-sulfonic acid and salts thereof;
sulfonic acid derivatives of 3-benzylidene camphor such as, for example, 4-(2-oxo-3-bornylidenemethyl)-benzene sulfonic acid and 2-methyl-5-(2-oxo-3-bornylidene)-sulfonic acid and salts thereof.

Typical UV-A filters are, in particular, derivatives of benzoyl methane such as, for example, 1-(4′-tert.butylphenyl)-3-(4′-methoxyphenyl)-propane-1,3-dione, 4-tert.butyl-4′-methoxydibenzoyl methane (Parsol 1789) or 1-phenyl-3-(4′-isopropylphenyl)-propane-1,3-dione and the enamine compounds described in DE 197 12 033 A1 (BASF). The UV-A and UV-B filters may of course also be used in the form of mixtures. Besides the soluble substances mentioned, insoluble light-blocking pigments, i.e. finely dispersed metal oxides or salts, may also be used for this purpose. Examples of suitable metal oxides are, in particular, zinc oxide and titanium dioxide and also oxides of iron, zirconium oxide, silicon, manganese, aluminium and cerium and mixtures thereof. Silicates (talcum), barium sulfate and zinc stearate may be used as salts. The oxides and salts are used in the form of the pigments for skin-care and skin-protecting emulsions and decorative cosmetics. The particles should have a mean diameter of less than 100 nm, preferably between 5 and 50 nm and more preferably between 15 and 30 nm. They may be spherical in shape although ellipsoidal particles or other non-spherical particles may also be used. The pigments may also be surface-treated, i.e. hydrophilicized or hydrophobicized. Typical examples are coated titanium dioxides, for example Titandioxid T 805 (Degussa) and Eusolex® T2000 (Merck). Suitable hydrophobic coating materials are, above all, silicones and, among these, especially trialkoxyoctylsilanes or dimethicones. So-called micro- or nanopigments are preferably used in sun protection products. Micronized zinc oxide is preferably used. Other suitable UV filters can be found in P. Finkel's review in SÖFW-Journal 122, 543 (1996).

Besides the two groups of primary sun protection factors mentioned above, secondary sun protection factors of the antioxidant type may also be used. Secondary sun protection factors of the antioxidant type interrupt the photochemical reaction chain which is initiated when UV rays penetrate into the skin. Typical examples are amino acids (for example glycine, histidine, tyrosine, tryptophane) and derivatives thereof, imidazoles (for example urocanic acid) and derivatives thereof, peptides, such as D,L-carnosine, D-carnosine, L-carnosine and derivatives thereof (for example anserine), carotinoids, carotenes (for example α-carotene, β-carotene, lycopene, lutein) and derivatives thereof, chlorogenic acid and derivatives thereof, liponic acid and derivatives thereof (for example dihydroliponic acid), aurothioglucose, propylthiouracil and other thiols (for example thioredoxine, glutathione, cysteine, cystine, cystamine and glycosyl, N-acetyl, methyl, ethyl, propyl, amyl, butyl and lauryl, palmitoyl, oleyl, γ-linoleyl, cholesteryl and glyceryl esters thereof) and their salts, dilaurylthiodipropionate,. distearylthiodipropionate, thiodipropionic acid and derivatives thereof (esters, ethers, peptides, lipids, nucleotides, nucleosides and salts) and sulfoximine compounds (for example butionine sulfoximines, homocysteine sulfoximine, butionine sulfones, penta-, hexa- and hepta-thionine sulfoximine) in very small compatible dosages (for example pmole to μmole/kg), also (metal) chelators (for example α-hydroxyfatty acids, palmitic acid, phytic acid, lactoferrine), α-hydroxy acids (for example citric acid, lactic acid, malic acid), humic acid, bile acid, bile extracts, bilirubin, biliverdin, EDTA, EGTA and derivatives thereof, unsaturated fatty acids and derivatives thereof (for example γ-linolenic acid, linoleic acid, oleic acid), folic acid and derivatives thereof, ubiquinone and ubiquinol and derivatives thereof, vitamin C and derivatives thereof (for example ascorbyl palmitate, Mg ascorbyl phosphate, ascorbyl acetate), tocopherols and derivatives (for example vitamin E acetate), vitamin A and derivatives (vitamin A palmitate) and coniferyl benzoate of benzoin resin, rutinic acid and derivatives thereof, α-glycosyl rutin, ferulic acid, furfurylidene glucitol, carnosine, butyl hydroxytoluene, butyl hydroxyanisole, nordihydroguaiac resin acid, nordihydroguaiaretic acid, trihydroxybutyrophenone, uric acid and derivatives thereof, mannose and derivatives thereof, Superoxid-Dismutase, zinc and derivatives thereof (for example ZnO, ZnSO₄), selenium and derivatives thereof (for example selenium methionine), stilbenes and derivatives thereof (for example stilbene oxide, trans-stilbene oxide) and derivatives of these active substances suitable for the purposes of the invention (salts, esters, ethers, sugars, nucleotides, nucleosides, peptides and lipids).

In addition, hydrotropes, for example ethanol, isopropyl alcohol or polyols, may be used to improve flow behavior. Suitable polyols preferably contain 2 to 15 carbon atoms and at least two hydroxyl groups. The polyols may contain other functional groups, more especially amino groups, or may be modified with nitrogen. Typical examples are
glycerol;
alkylene glycols such as, for example, ethylene glycol, diethylene glycol, propylene glycol, butylene glycol, hexylene glycol and polyethylene glycols with an average molecular weight of 100 to 1000 dalton;
technical oligoglycerol mixtures with a degree of self-condensation of 1.5 to 10 such as, for example, technical diglycerol mixtures with a diglycerol content of 40 to 50% by weight;
methylol compounds such as, in particular, trimethylol ethane, trimethylol propane, trimethylol butane, pentaerythritol and dipentaerythritol;
lower alkyl glucosides, particularly those containing 1 to 8 carbon atoms in the alkyl group, for example methyl and butyl glucoside;
sugar alcohols containing 5 to 12 carbon atoms, for example sorbitol or mannitol,
sugars containing 5 to 12 carbon atoms, for example glucose or sucrose;
amino sugars, for example glucamine;
dialcoholamines, such as diethanolamine or 2-aminopropane-1,3-diol.

Suitable preservatives are, for example, phenoxyethanol, formaldehyde solution, parabens, pentanediol or sorbic acid and the other classes of compounds listed in Appendix 6, Parts A and B of the Kosmetikverordnung (“Cosmetics Directive”). Suitable insect repellents are N,N-diethyl-m-toluamide, pentane-1,2-diol or Ethyl Butylacetyl-aminopropionate. A suitable self-tanning agent is dihydroxyacetone. Suitable tyrosine inhibitors which prevent the formation of melanin and are used in depigmenting agents are, for example, arbutin, ferulic acid kojic acid, coumaric acid and ascorbic acid (vitamin C).

Suitable perfume oils are mixtures of natural and synthetic perfumes. Natural perfumes include the extracts of blossoms (lily, lavender, rose, jasmine, neroli, ylang-ylang), stems and leaves (geranium, patchouli, petitgrain), fruits (anise, coriander, caraway, juniper), fruit peel (bergamot, lemon, orange), roots (nutmeg, angelica, celery, cardamon, costus, iris, calmus), woods (pinewood, sandalwood, guaiac wood, cedarwood, rosewood), herbs and grasses (tarragon, lemon grass, sage, thyme), needles and branches (spruce, fir, pine, dwarf pine), resins and balsams (galbanum, elemi, benzoin, myrrh, olibanum, opoponax). Animal raw materials, for example civet and beaver, may also be used. Typical synthetic perfume compounds are products of the ester, ether, aldehyde, ketone, alcohol and hydrocarbon type. Examples of perfume compounds of the ester type are benzyl acetate, phenoxyethyl isobutyrate, p-tert.butyl cyclohexylacetate, linalyl acetate, dimethyl benzyl carbinyl acetate, phenyl ethyl acetate, linalyl benzoate, benzyl formate, ethylmethyl phenyl glycinate, allyl cyclohexyl propionate, styrallyl propionate and benzyl salicylate. Ethers include, for example, benzyl ethyl ether while aldehydes include, for example, the linear alkanals containing 8 to 18 carbon atoms, citral, citronellal, citronellyloxyacetaldehyde, cyclamen aldehyde, hydroxycitronellal, lilial and bourgeonal. Examples of suitable ketones are the ionones, α-isomethylionone and methyl cedryl ketone. Suitable alcohols are anethol, citronellol, eugenol, isoeugenol, geraniol, linalool, phenylethyl alcohol and terpineol. The hydrocarbons mainly include the terpenes and balsams. However, it is preferred to use mixtures of different perfume compounds which, together, produce an agreeable fragrance. Other suitable perfume oils are essential oils of relatively low volatility which are mostly used as aroma components. Examples are sage oil, camomile oil, clove oil, melissa oil, mint oil, cinnamon leaf oil, lime-blossom oil, juniper berry oil, vetiver oil, olibanum oil, galbanum oil, ladanum oil and lavendin oil. The following are preferably used either individually or in the form of mixtures: bergamot oil, dihydromyrcenol, lilial, lyral, citronellol, phenylethyl alcohol, α-hexylcinnamaldehyde, geraniol, benzyl acetone, cyclamen aldehyde, linalool, Boisambrene Forte, Ambroxan, indole, hedione, sandelice, citrus oil, mandarin oil, orange oil, allylamyl glycolate, cyclovertal, lavendin oil, clary oil, β-damascone, geranium oil bourbon, cyclohexyl salicylate, Vertofix Coeur, Iso-E-Super, Fixolide NP, evernyl, iraldein gamma, phenylacetic acid, geranyl acetate, benzyl acetate, rose oxide, romillat, irotyl and floramat.

Suitable dyes are any of the substances suitable and approved for cosmetic purposes as listed, for example, in the publication “Kosmetische Färbemittel” of the Farbstoffkommission der Deutschen Forschungs-gemeinschaft, Verlag Chemie, Weinheim, 1984, pages 81 to 106. These dyes are normally used in concentrations of 0.001 to 0.1% by weight, based on the mixture as a whole.

The total percentage content of auxiliaries and additives may be from 1 to 50% by weight and is preferably from 5 to 40% by weight, based on the particular preparation. The preparations may be produced by standard hot or cold processes and are preferably produced by the phase inversion temperature method.

Table 1 below presents a number of Formulation Examples using a residue from the production of red Bordeaux wine which contained 4% by weight polyphenols and 35% by weight of proteins, based on the dry residue.

### EXAMPLES

TABLE 1
Cosmetic preparations (water, preservative to 100% by weight)

| Composition (INCI) | 1 | 2 | 3 | 4 | 5 | 6 | 7 | 8 | 9 | 10 |
|---|---|---|---|---|---|---|---|---|---|---|
| Texapon ® NSO | — | — | — | — | — | — | 38.0 | 38.0 | 25.0 | — |
| Sodium Laureth Sulfate | | | | | | | | | | |
| Texapon ® SB 3 | — | — | — | — | — | — | — | — | 10.0 | — |
| Disodium Laureth Sulfosuccinate | | | | | | | | | | |
| Plantacare ® 818 | — | — | — | — | — | — | 7.0 | 7.0 | 6.0 | — |
| Coca Glucosides | | | | | | | | | | |
| Plantacare ® PS 10 | — | — | — | — | — | — | — | — | — | 16.0 |
| Sodium Laureth Sulfate (and) Coco Glucosides | | | | | | | | | | |
| Dehyton ® PK 45 | — | — | — | — | — | — | — | — | 10.0 | — |
| Cocamidopropyl Betaine | | | | | | | | | | |
| Dehyquart ® A | 2.0 | 2.0 | 2.0 | 2.0 | 4.0 | 4.0 | — | — | — | — |
| Cetrimonium Chloride | | | | | | | | | | |
| Dehyquart L ® 80 | 1.2 | 1.2 | 1.2 | 1.2 | 0.6 | 0.6 | — | — | — | — |
| Dococoylmethylethoxymonium Methosulfate (and) | | | | | | | | | | |
| Propyleneglycol | | | | | | | | | | |
| Eumulgin ® B2 | 0.8 | 0.8 | — | 0.8 | — | 1.0 | — | — | — | — |
| Ceteareth-20 | | | | | | | | | | |
| Eumulgin ® VL 75 | — | — | 0.8 | — | 0.8 | — | — | — | — | — |
| Lauryl Glucoside (and) Polyglyceryl-2 Polyhydroxystearate | | | | | | | | | | |
| (and) Glycerin | | | | | | | | | | |
| Lanette ® O | 2.5 | 2.5 | 2.5 | 2.5 | 3.0 | 2.5 | — | — | — | — |
| Cetearyl Alcohol | | | | | | | | | | |
| Cutina ® GMS | 0.5 | 0.5 | 0.5 | 0.5 | 0.5 | 1.0 | — | — | — | — |
| Glyceryl Stearate | | | | | | | | | | |
| Cetiol ® HE | 1.0 | — | — | — | — | — | — | — | 1.0 | |
| PEG-7 Glyceryl Cocoate | | | | | | | | | | |
| Cetiol ® PGL | — | 1.0 | — | — | 1.0 | — | — | — | — | — |
| Hexyldecanol (and) Hexyldecyl laurate | | | | | | | | | | |
| Cetiol ® V | — | — | — | 1.0 | — | — | — | — | — | — |
| Decyl Oleate | | | | | | | | | | |
| Eutanol ® G | — | — | 1.0 | — | — | 1.0 | — | — | — | — |
| Octyldodecanol | | | | | | | | | | |
| Nutrilan ® Keratin W | — | — | — | 2.0 | — | — | — | — | — | — |
| Hydrolyzed Keratin | | | | | | | | | | |
| Lamesoft ® LMG | — | — | — | — | — | — | 3.0 | 2.0 | 4.0 | — |
| Glyceryl Laurate (and) Potassium Cocoyl Hydrolyzed | | | | | | | | | | |
| Collagen | | | | | | | | | | |
| Euperlan ® PK 3000 AM | — | — | — | — | — | — | — | 3.0 | 5.0 | 5.0 |
| Glycol Distearate (and) Laureth-4 (and) Cocamidopropyl | | | | | | | | | | |
| Betaine | | | | | | | | | | |
| Generol ® 122 N | — | — | — | — | 1.0 | 1.0 | — | — | — | — |
| Soya Sterol | | | | | | | | | | |
| Residue containing polyphenols and | 1.0 | 1.0 | 1.0 | 1.0 | 1.0 | 1.0 | 1.0 | 1.0 | 1.0 | 1.0 |
| proteins | | | | | | | | | | |
| Hydagen ® HCMF | 1.0 | 1.0 | 1.0 | 1.0 | 1.0 | 1.0 | 1.0 | 1.0 | 1.0 | 1.0 |
| Chitosan | | | | | | | | | | |
| Copherol ® 12250 | — | — | 0.1 | 0.1 | — | — | — | — | — | — |
| Tocopherol Acetate | | | | | | | | | | |
| Arlypon ® F | — | — | — | — | — | — | 3.0 | 3.0 | 1.0 | — |
| Laureth-2 | | | | | | | | | | |
| Sodium Chloride | — | — | — | — | — | — | — | 1.5 | — | 1.5 |

(1-4) hair rinse,
(5-6) conditioner,
(7-8) shower bath,
(9) shower gel,
(10) wash lotion

| Composition (INCI) | 11 | 12 | 13 | 14 | 15 | 16 | 17 | 18 | 19 | 20 |
|---|---|---|---|---|---|---|---|---|---|---|
| Texapon ® NSO | 20.0 | 20.0 | 12.4 | — | 25.0 | 11.0 | — | — | — | — |
| Sodium Laureth Sulfate | | | | | | | | | | |
| Texpon ® K 14 S | — | — | — | — | — | — | — | — | 11.0 | 23.0 |
| Sodium Myreth Sulfate | | | | | | | | | | |
| Texapon ® SB 3 | — | — | — | — | — | 7.0 | — | — | — | — |
| Disodium Laureth Sulfosuccinate | | | | | | | | | | |
| Plantacare ® 818 | 5.0 | 5.0 | 4.0 | — | — | — | — | — | 6.0 | 4.0 |
| Coca Glucosides | | | | | | | | | | |
| Plantacare ® 2000 | — | — | — | — | 5.0 | 4.0 | — | — | — | — |
| Decyl Glucoside | | | | | | | | | | |
| Plantacare ® PS 10 | — | — | — | 40.0 | — | — | 16.0 | 17.0 | — | — |
| Sodium Laureth Sulfate (and) Coco Glucosides | | | | | | | | | | |
| Dehyton ® PK 45 | 20.0 | 20.0 | — | — | 8.0 | — | — | — | — | 7.0 |
| Cocamidopropyl Betaine | | | | | | | | | | |
| Eumulgin ® B1 | — | — | — | — | 1.0 | — | — | — | — | — |
| Ceteareth-12 | | | | | | | | | | |
| Eumulgin ® B2 | — | — | — | 1.0 | — | — | — | — | — | — |
| Ceteareth-20 | | | | | | | | | | |
| Lameform ® TGI | — | — | — | 4.0 | — | — | — | — | — | — |
| Polyglyceryl-3 Isostearate | | | | | | | | | | |
| Dehymuls ® PGPH | — | — | 1.0 | — | — | — | — | — | — | — |
| Polyglyceryl-2 Dipolyhydroxystearate | | | | | | | | | | |
| Monomuls ® 90-L 12 | — | — | — | — | — | — | — | — | 1.0 | 1.0 |
| Glyceryl Laurate | | | | | | | | | | |
| Cetiol ® HE | — | 0.2 | — | — | — | — | — | — | — | — |
| PEG-7 Glyceryl Cocoate | | | | | | | | | | |
| Eutanol ® G | — | — | — | 3.0 | — | — | — | — | — | — |
| Octyldodecanol | | | | | | | | | | |
| Nutrilan ® Keratin W | — | — | — | — | — | — | — | — | 2.0 | 2.0 |
| Hydrolyzed Keratin | | | | | | | | | | |
| Nutrilan ® I | 1.0 | — | — | — | — | 2.0 | — | 2.0 | — | — |
| Hydrolyzed Collagen | | | | | | | | | | |
| Lamesoft ® LMG | — | — | — | — | — | — | — | — | 1.0 | — |
| Glyceryl Laurate (and) Potassium Cocoyl | | | | | | | | | | |
| Hydrolyzed Collagen | | | | | | | | | | |
| Lamesoft ® 156 | — | — | — | — | — | — | — | — | — | 5.0 |
| Hydrogenated Tallow Glyceride (and) | | | | | | | | | | |
| Potassium Cocoyl Hyrolyzed Collagen | | | | | | | | | | |
| Gluadin ® WK | 1.0 | 1.5 | 4.0 | 1.0 | 3.0 | 1.0 | 2.0 | 2.0 | 2.0 | — |
| Sodium Cocoyl Hydrolyzed Wheat Protein | | | | | | | | | | |
| Euperlan ® PK 3000 AM | 5.0 | 3.0 | 4.0 | — | — | — | — | 3.0 | 3.0 | — |
| Glycol Distearate (and) Laureth-4 (and) | | | | | | | | | | |
| Cocamidopropyl Betaine | | | | | | | | | | |
| Panthenol | — | — | 1.0 | — | — | — | — | — | — | — |
| Arlypon ® F | 2.6 | 1.6 | — | 1.0 | 1.5 | — | — | — | — | — |
| Laureth-2 | | | | | | | | | | |
| Residue containing polyphenols and | 1.0 | 1.0 | 1.0 | 1.0 | 1.0 | 1.0 | 1.0 | 1.0 | 1.0 | 1.0 |
| proteins | | | | | | | | | | |
| Hydagen ® CMF | 1.0 | 1.0 | 1.0 | 1.0 | 1.0 | 1.0 | 1.0 | 1.0 | 1.0 | 1.0 |
| Chitosan | | | | | | | | | | |
| Sodium Chloride | — | — | — | — | — | 1.6 | 2.0 | 2.2 | — | 3.0 |
| Glycerin (86% by weight) | — | 5.0 | — | — | — | — | — | 1.0 | 3.0 | — |

(11-14) “two-in-one” shower bath,
(15-20) shampoo

| Composition (INCI) | 21 | 22 | 23 | 24 | 25 | 26 | 27 | 28 | 29 | 30 |
|---|---|---|---|---|---|---|---|---|---|---|
| Texapon ® NSO | — | 30.0 | 30.0 | — | 25.0 | — | — | — | — | — |
| Sodium Laureth Sulfate | | | | | | | | | | |
| Plantacare ® 818 | — | 10.0 | — | — | 20.0 | — | — | — | — | — |
| Coco Glucosides | | | | | | | | | | |
| Plantacare ® PS 10 | 22.0 | — | 5.0 | 22.0 | — | — | — | — | — | — |
| Sodium Laureth Sulfate (and) Coco Glucosides | | | | | | | | | | |
| Dehyton ® PK 45 | 15.0 | 10.0 | 15.0 | 15.0 | 20.0 | — | — | — | — | — |
| Cocamidopropyl Betaine | | | | | | | | | | |
| Emulgade ® SE | — | — | — | — | — | 5.0 | 5.0 | 4.0 | — | — |
| Glyceryl Stearate (and) Ceteareth 12/20 | | | | | | | | | | |
| (and) Cetearyl Alcohol (and) Cetyl Palmitate | | | | | | | | | | |
| Eumulgin ® B1 | — | — | — | — | — | — | — | 1.0 | — | — |
| Ceteareth-12 | | | | | | | | | | |
| Lameform ® TGI | — | — | — | — | — | — | — | — | 4.0 | — |
| Polyglyceryl-3 Isostearate | | | | | | | | | | |
| Dehymuls ® PGPH | — | — | — | — | — | — | — | — | — | 4.0 |
| Polyglyceryl-2 Dipolyhydroxystearate | | | | | | | | | | |
| Monomuls ® 90-O 18 | — | — | — | — | — | — | — | — | 2.0 | — |
| Glyceryl Oleate | | | | | | | | | | |
| Cetiol ® HE | 2.0 | — | — | 2.0 | 5.0 | — | — | — | — | 2.0 |
| PEG-7 Glyceryl Cocoate | | | | | | | | | | |
| Cetiol ® OE | — | — | — | — | — | — | — | — | 5.0 | 6.0 |
| Dicaprylyl Ether | | | | | | | | | | |
| Cetiol ® PGL | — | — | — | — | — | — | — | 3.0 | 10.0 | 9.0 |
| Hexyldecanol (and) Hexyldecyl Laurate | | | | | | | | | | |
| Cetiol ® SN | — | — | — | — | — | 3.0 | 3.0 | — | — | — |
| Cetearyl Isononanoate | | | | | | | | | | |
| Cetiol ® V | — | — | — | — | — | 3.0 | 3.0 | — | — | — |
| Decyl Oleate | | | | | | | | | | |
| Myritol ® 318 | — | — | — | — | — | — | — | 3.0 | 5.0 | 5.0 |
| Coco Caprylate Caprate | | | | | | | | | | |
| Bees Wax | — | — | — | — | — | — | — | — | 7.0 | 5.0 |
| Nutrilan ® Elastin E20 | — | — | — | — | — | 2.0 | — | — | — | — |
| Hydrolyzed Elastin | | | | | | | | | | |
| Nutrilan ® I-50 | — | — | — | — | 2.0 | — | 2.0 | — | — | — |
| Hydrolyzed Collagen | | | | | | | | | | |
| Gluadin ® AGP | 0.5 | 0.5 | 0.5 | — | — | — | — | 0.5 | — | — |
| Hydrolyzed Wheat Gluten | | | | | | | | | | |
| Gluadin ® WK | 2.0 | 2.0 | 2.0 | 2.0 | 5.0 | — | — | — | 0.5 | 0.5 |
| Sodium Cocoyl Hydrolyzed Wheat Protein | | | | | | | | | | |
| Euperlan ® PK 3000 AM | 5.0 | — | — | 5.0 | — | — | — | — | — | — |
| Glycol Distearate (and) Laureth-4 (and) Cocamidopropyl | | | | | | | | | | |
| Betaine | | | | | | | | | | |
| Arlypon ® F | — | — | — | — | — | — | — | — | — | — |
| Laureth-2 | | | | | | | | | | |
| Residue containing polyphenols and | 1.0 | 1.0 | 1.0 | 1.0 | 1.0 | 1.0 | 1.0 | 1.0 | 1.0 | 1.0 |
| proteins | | | | | | | | | | |
| Hydagen ® CMF | 1.0 | 1.0 | 1.0 | 1.0 | 1.0 | 1.0 | 1.0 | 1.0 | 1.0 | 1.0 |
| Chitosan | | | | | | | | | | |
| Magnsium Sulfate Hepta Hydrate | — | — | — | — | — | — | — | — | 1.0 | 1.0 |
| Glycerin (85% by weight) | — | — | — | — | — | 3.0 | 3.0 | 5.0 | 5.0 | 3.0 |

(21-25) foam bath,
(26) soft cream,
(27, 28) moisturising emulsion,
(29, 30) wash lotion

| Composition (INCI) | 31 | 32 | 33 | 34 | 35 | 36 | 37 | 38 | 39 | 40 |
|---|---|---|---|---|---|---|---|---|---|---|
| Dehymuls ® PGPH | 4.0 | 3.0 | — | 5.0 | — | — | — | — | — | — |
| Polyglyceryl-2 Dipolyhydroxystearate | | | | | | | | | | |
| Lameform ® TGI | 2.0 | 1.0 | — | — | — | — | — | — | — | — |
| Polyglyceryl-3 Diisostearate | | | | | | | | | | |
| Emulgade ® PL 68/50 | — | — | — | — | 4.0 | — | — | — | 3.0 | — |
| Cetearyl Glucoside (and) Cetearyl Alcohol | | | | | | | | | | |
| Eumulgin ® B2 | — | — | — | — | — | — | — | 2.0 | — | — |
| Ceteareth-20 | | | | | | | | | | |
| Tegocare ® PS | — | — | 3.0 | — | — | — | 4.0 | — | — | — |
| Polyglyceryl-3 Methylglucose Distearate | | | | | | | | | | |
| Eumulgin VL 75 | — | — | — | — | — | 3.5 | — | — | 2.5 | — |
| Polyglyceryl-2 Dipolyhydroxystearate (and) Lauryl Glucoside | | | | | | | | | | |
| (and) Glycerin | | | | | | | | | | |
| Bees Wax | 3.0 | 2.0 | 5.0 | 2.0 | — | — | — | — | — | — |
| Cutina ® GMS | — | — | — | — | — | 2.0 | 4.0 | — | — | 4.0 |
| Glyceryl Stearate | | | | | | | | | | |
| Lanette ® O | — | — | 2.0 | — | 2.0 | 4.0 | 2.0 | 4.0 | 4.0 | 1.0 |
| Cetearyl Alcohol | | | | | | | | | | |
| Antaron ® V 216 | — | — | — | — | — | 3.0 | — | — | — | 2.0 |
| PVP/Hexadecene Copolymer | | | | | | | | | | |
| Myritol ® 818 | 5.0 | — | 10.0 | — | 8.0 | 6.0 | 6.0 | — | 5.0 | 5.0 |
| Cocoglycerides | | | | | | | | | | |
| Finsolv ® TN | — | 6.0 | — | 2.0 | — | — | 3.0 | — | — | 2.0 |
| C12/15 Alkyl Benzoate | | | | | | | | | | |
| Cetiol ® J 600 | 7.0 | 4.0 | 3.0 | 5.0 | 4.0 | 3.0 | 3.0 | — | 5.0 | 4.0 |
| Oleyl Erucate | | | | | | | | | | |
| Cetiol ® OE | 3.0 | — | 6.0 | 8.0 | 6.0 | 5.0 | 4.0 | 3.0 | 4.0 | 6.0 |
| Dicaprylyl Ether | | | | | | | | | | |
| Mineral Oil | — | 4.0 | — | 4.0 | — | 2.0 | — | 1.0 | — | — |
| Cetiol ® PGL | — | 7.0 | 3.0 | 7.0 | 4.0 | — | — | — | 1.0 | — |
| Hexadecanol (and) Hexyldecyl Laurate | | | | | | | | | | |
| Panthenol/Bisabolol | 1.2 | 1.2 | 1.2 | 1.2 | 1.2 | 1.2 | 1.2 | 1.2 | 1.2 | 1.2 |
| Residue containing polyphenols and | 1.0 | 1.0 | 1.0 | 1.0 | 1.0 | 1.0 | 1.0 | 1.0 | 1.0 | 1.0 |
| proteins | | | | | | | | | | |
| Hydagen ® CMF | 1.0 | 1.0 | 1.0 | 1.0 | 1.0 | 1.0 | 1.0 | 1.0 | 1.0 | 1.0 |
| Chitosan | | | | | | | | | | |
| Copherol ® F 1300 | 0.5 | 1.0 | 1.0 | 2.0 | 1.0 | 1.0 | 1.0 | 2.0 | 0.5 | 2.0 |
| Tocopherol/Tocopheryl Acetate | | | | | | | | | | |
| Neo Heliopan ® Hydro | 3.0 | — | — | 3.0 | — | — | 2.0 | — | 2.0 | — |
| Sodium Phenylbenzimidazole Sulfonate | | | | | | | | | | |
| Neo Heliopan ® 303 | — | 5.0 | — | — | — | 4.0 | 5.0 | — | — | 10.0 |
| Octocrylene | | | | | | | | | | |
| Neo Heliopan ® BB | 1.5 | — | — | 2.0 | 1.5 | — | — | — | 2.0 | — |
| Benzophenone-3 | | | | | | | | | | |
| Neo Heliopan ® E 1000 | 5.0 | — | 4.0 | — | 2.0 | 2.0 | 4.0 | 10.0 | — | — |
| Isoamyl p-Methoxycinnamate | | | | | | | | | | |
| Neo Heliopan ® AV | 4.0 | — | 4.0 | 3.0 | 2.0 | 3.0 | 4.0 | — | 10.0 | 2.0 |
| Octyl Methoxycinnamate | | | | | | | | | | |
| Uvinul ® T 150 | 2.0 | 4.0 | 3.0 | 1.0 | 1.0 | 1.0 | 4.0 | 3.0 | 3.0 | 3.0 |
| Octyl Triazone | | | | | | | | | | |
| Zinc Oxide | — | 6.0 | 6.0 | — | 4.0 | — | — | — | — | 5.0 |
| Titanium Dioxide | — | — | — | — | — | — | — | 5.0 | — | — |
| Glycerol (86% by weight) | 5.0 | 5.0 | 5.0 | 5.0 | 5.0 | 5.0 | 5.0 | 5.0 | 5.0 | 5.0 |

(31) w/o sun protection cream,
(32-34) w/o sun protection lotion,
(35, 38, 40) o/w sun protection lotion,
(36, 27, 39) o/w sun protection cream

We claim:

1-14. (canceled)

15. A method for countering effects of at least one of skin aging and damage to fibroblasts and/or keratinocytes by UV-A and UV-B radiation which comprises applying to an area of the skin, in which the effects are to be countered, an active composition comprising residues of wine production comprising 1% to 10% by weight of polyphenols and 25% to 50% by weight of proteins, based on a dry residue.

16. The method of claim 15, wherein, the wine residues comprise association complexes of polyphenols and proteins as active ingredients.

17. The method of claim 15, wherein, the residues of wine production comprise at least one member selected from the group consisting of proteins from the cell membrane of yeast and enzyme degradation products of Saccharomyces cerevisiae type.

18. The method of claim 15, wherein, the active composition contains from 0.1% to 100% of the residues of wine production.

19. The method of claim 16, wherein, the active composition contains from 0.1% to 100% of the residues of wine production.

20. The method of claim 17, wherein, the active composition contains from 0.1% to 100% of the residues of wine production.

21. The method of claim 16, wherein, the residues of wine production comprise at least one member selected from the group consisting of proteins from the cell membrane of yeast and enzyme degradation products of Saccharomyces cerevisiae type.
